(19) United States
(12) Patent Application Publication
(10) Pub. No.: US 2023/0210591 A1
(43) Pub. Date: Jul. 6, 2023

(54) TEMPERATURE SENSING CATHETER

(71) Applicants: Alexander MCLELLAN, Malvern East, Victoria (AU); Elli TUTUNGI, Malvern, Victoria (AU); Matthew HALLAM, Highett, Victoria (AU); Vicki TUTUNGI, Malvern, Victoria (AU)

(72) Inventors: Alexander MCLELLAN, Malvern East, Victoria (AU); Elli TUTUNGI, Malvern, Victoria (AU); Matthew HALLAM, Highett, Victoria (AU); Vicki TUTUNGI, Malvern, Victoria (AU)

(21) Appl. No.: 17/996,671

(22) PCT Filed: Apr. 21, 2021
(86) PCT No.: PCT/IB2021/000265

§ 371 Date: Oct. 20, 2022

Related U.S. Application Data
(60) Provisional application No. 63/013,495, filed on Apr. 21, 2020.

(51) Int. Cl. A61B 18/14; A61B 5/01; A61B 5/287

(52) U.S. Cl. CPC A61B 18/1492; A61B 5/015; A61B 5/287; A61B 2018/00351

(57) ABSTRACT

Temperature sensing catheters and systems that can be used during cardiac ablation procedures to measure and monitor temperatures, and the rate and spread of temperature changes in the heart. The temperature data can be used to calculate temperature gradients, which may be used to estimate if and when certain regions of heart may undergo injury due to thermal exposure. The temperature data can be used to limit or cut-off power delivery to an ablation catheter, or otherwise modify the ablation procedure, to prevent injury to certain regions of heart. In some cases, the temperature data is used to control aspects of the ablation in a feedback loop control scheme.

### CROSS REFERENCE TO RELATED APPLICATIONS

This patent application claims priority to U.S. Provisional Patent Application No. 63/013,495, titled “TEMPERATURE SENSING CATHETER,” and filed on Apr. 21, 2020.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The devices, systems and methods described herein relate to cardiac ablation procedures. Cardiac catheters for measuring temperature of heart tissue and methods of their use during cardiac ablation are described.

### BACKGROUND

The heart beat in a healthy human is controlled by the sinoatrial node (SA node) located in the right atrium. The SA node generates electrical signal potentials that are transmitted through pathways of conductive heart tissue in the atrium to the atrioventricular node (AV node), which in turn transmits the electrical signals to the ventricle by means of the His and Purkinje conductive tissues. Abnormalities to the conductive tissue in the heart can interfere with the passage of regular electrical signals from the SA node or through the AV node. Electrical signal irregularities resulting from such interference can disturb the normal rhythm of the heart and cause an abnormal rhythmic condition referred to as cardiac arrhythmia.

Electrophysiological ablation is a procedure often successful in treating cardiac arrhythmia. This procedure involves applying sufficient energy to the interfering tissue to ablate that tissue thus removing the causative circuit or focus. The ablation procedure requires location of the interfering tissue which in some cases is close to the AV node. Care must be taken during the ablation procedure to ensure that the AV node itself is not damaged during the ablation. If the AV node is irreversibly damaged the patient may require a permanent pacemaker to restore atrioventricular synchrony.

Electrophysiological mapping can be used to identify the interfering tissue using a series of diagnostic electrophysiology (EP) catheters as well as the treating catheter (e.g., using ablation). The electrical signals emanating from the endocardial tissues are systematically monitored and sometimes incorporated into a three-dimensional map. By analyzing the signals or map, the interfering electrical focus or circuit can be identified. The conventional method for mapping the electrical signals from conductive heart tissue is to percutaneously introduce a series of electrophysiology (EP) catheters having intracardiac electrogram mapping (EGM) electrodes configured to detect electrical activity and mounted on their distal extremity. The catheters are maneuvered to place the EGM electrodes in contact with or in close proximity to the endocardium of the patient's heart. By monitoring the electrical signals at the endocardium, tissue critically participating in the arrhythmia can be pinpointed.

Once the origin of the arrhythmia is located in the tissue, the physician may use an ablation procedure to destroy the tissue causative or critically participating in the arrhythmia, with the goal of restoring normal rhythm, making the arrhythmia non-inducible, and not damaging the normal conduction system (e.g., the AV node). This procedure is typically done under guidance by fluoroscopy and sometimes utilizing a three-dimensional mapping system to visualize the diagnostic and/or ablation catheters.

The treating catheter is called an ablation catheter and routinely destroys or removes tissue through the use of thermal injury to create a scar at the treatment site. The most common ablation catheters deliver radiofrequency energy or cryothermal energy. Radiofrequency (RF) catheters create lesions through conversion of alternating electrical energy to thermal energy by a combination or resistive and conductive heating; tissue becomes non-viable at temperatures approximating 50° C. Cryotherapy catheters make tissue reversibly impaired at −30° C. and irreversibly impaired at approximately −50° C.

Many arrhythmias are in close proximity to the AV node and there is the potential that while ablating the pathological circuit, the AV node may be inadvertently damaged. The standard size of ablation lesions is approximately five millimeters, though with longer duration ablation, irrigated ablation and/or higher power ablation, the lesion size may increase. Little is known about the spread of the thermal wave during ablation procedures, particularly in relation to the AV node.

Temperature sensors have been incorporated in ablation (treatment) catheters for some time. These catheters measure the local temperature at the ablation catheter's site only. Ablation catheters generally have a temperature sensor guiding ablation by limiting power delivery to the catheter when temperature exceeds a pre-specified set-point (usually 60° C. for solid tip and 40° C. for irrigated radiofrequency ablation catheters). One reason to measure and limit ablation catheter temperature is for the recognized phenomenon of temperature at the ablation site approximating 100° C. causing formation of gas bubbles that may burst and cause barotrauma (steam pop), that may lead to significant cardiac injury including cardiac perforation.

Currently there is no system or catheter where a diagnostic (non-ablation) catheter incorporates a temperature sensor (or series of temperature sensors) to measure temperature at the AV node or anywhere distant to the local site of ablation, and potentially forewarn and advert damage to the AV node. Hence, there is a recognized need for a temperature sensing system for protecting the AV node and/or other portions of the heart. There is also a recognized need for such a system that does not significantly increase the expense of the procedure yet reduces the risk of damaging the AV node.

### SUMMARY

Described herein are devices, systems and methods for monitoring temperature of one or more regions of the heart during a cardiac ablation procedure. The temperature measurements may be detected a distance from the ablation site and near (or at) a region of the heart susceptible to injury from exposure to heat or cold during the ablation process. Temperature monitoring may be done using a temperature sensing catheter that includes multiple temperature sensors along the catheter body for recording temperatures of tissue at or near a region of interest during the ablation. The temperature sensing catheter may include mapping electrodes for positioning the temperature sensing catheter relative to the region of interest.

In some implementations, the methods are used to monitor temperature during an AV-nodal reentrant tachycardia (AVNRT) ablation procedure. In the case of AVNRT, the ablation site may include a slow pathway and/or a fast pathway exit of the AVNRT near the coronary sinus. The AV node may be susceptible to thermal injury during the ablation due to its proximity with respect to the typical ablation site for treating the AVNRT. If temperatures surpass an upper and/or lower limit, the AV node may undergo reversible or irreversible AV block. If temperatures of the AV node are estimated to reach or surpass the upper and/or lower limit, the user and/or an automated control system may be alerted to modify the ablation accordingly. In some cases, the temperature sensing catheter is positioned at or near the His bundle and/or the AV node to monitor the temperature at this region during the ablation procedure. In some embodiments, the temperature sensing catheter may be positioned at or near the coronary sinus. In some cases, two or more temperature sensing catheters are used to monitor multiple regions of the heart.

Temperature readings may include temperature gradient data based on differing temperatures among different temperature sensors of the catheter and their relative distances from the ablation site. The temperature gradient data can be used to calculate a number of parameters such as the rate of temperature, energy and/or power increases and decreases at the region of interest, and/or acceleration and deceleration of in temperature, energy, and/or power at the region of interest. This data can be compared to thresholds (e.g., upper and lower temperature, upper and lower rate of increase/decrease in temperature, and/or upper and lower temperature acceleration/deceleration) associated with causing injury to the region of interest. In some cases, the data is compared to multiple thresholds corresponding to different injury severities. The temperature sensing catheter may be configured to provide an alert (e.g., visible and/or audible alert) to the user to forewarn of the risk of injury.

In some cases, the temperature sensing catheter is configured to provide feedback data to a control system. For example, an ablation catheter system controlling the ablation process may be configured to receive feedback data from the temperature sensing catheter and automatically modify the ablation parameters accordingly. In some cases, the feedback data is used to halt or temporarily pause the ablation (e.g., turn off the heat or cold). In some cases, the feedback data is used to modify ablation parameters without stopping the ablation process, for example, by decreasing or increasing the intensity of the ablation.

These and other details, embodiments and advantages are described herein.

### BRIEF DESCRIPTION OF THE DRAWINGS

Novel features of embodiments described herein are set forth with particularity in the appended claims. A better understanding of the features and advantages of the embodiments may be obtained by reference to the following detailed description that sets forth illustrative embodiments and the accompanying drawings.

FIG. 1 illustrates a view of a heart showing the AV node and an AVNRT re-entrant circuit.

FIG. 2A illustrates an example temperature sensing catheter.

FIG. 2B illustrates a close-up view of a sensor portion of the temperature sensing catheter of FIG. 2A.

FIG. 2C is a graph indicating an example calculated rise in tissue temperature as a function of time for temperature sensors of the catheter of FIGS. 2A and 2B.

FIG. 2D is a graph comparing the calculated rise in tissue temperature measured at different temperature sensors of the catheter of FIGS. 2A and 2B compared to an estimated onset of injury to the AV node.

FIG. 2E is a graph indicating an example estimated time in which an AV block may occur based on measured temperatures using temperature sensors of the catheter of FIGS. 2A and 2B.

FIG. 3 illustrates a distal sensing portion of another example temperature sensing catheter.

FIG. 4A is a flowchart illustrating an example process for using a temperature sensing catheter during an ablation procedure.

FIG. 4B is a flowchart illustrating an example process for using a temperature sensing catheter during an ablation procedure including feedback.

FIG. 5A illustrates a simplified block diagram of a data processing system for a temperature sensing catheter system.

FIG. 5B illustrates a simplified block diagram of an example computing environment for a temperature sensing catheter system.

FIG. 6A-6G illustrate an example test setup and results from a test procedure: FIG. 6A illustrates a distal sensing portion of an example temperature sensing catheter used during the testing procedure; FIG. 6B illustrates cables for the temperature sensing catheter used during the test procedure; FIGS. 6C and 6D illustrate a test setup for the testing procedure; and FIGS. 6E-6G illustrate results from the test procedure.

FIGS. 7A-7D illustrate examples of temperature-sensing catheters, as described herein. FIG. 7A shows an example of a temperature-sensing cardiac catheter that can be deflected to include a first curved shape. FIG. 7B shows the catheter of FIG. 7A with the temperature sensors indicated. FIG. 7C shows a catheter such as the catheter of FIG. 7A-7B that can be controllably deflected to include multiple curves. FIG. 7D shows the catheter of FIG. 7C with the temperature sensors indicated.

FIGS. 7E and 7F illustrate one example of a temperature-sensitive catheter showing deflection into a curved shape having multiple curves. FIG. 7F shows a slightly enlarged view of the distal end of the catheter o FIG. 7E.

FIGS. 8A-8C illustrate another example of a temperature-sensing catheter configured as straight (e.g., linear) catheter including alternating electrodes and temperature sensors. FIG. 8A shows a side profile. FIG. 8B show a section through the elongate body of the device of FIG. 8A. FIG. 8C shows an enlarged view of the distal end of the catheter.

FIGS. 9A-9B illustrate an example of a temperature-sensing catheter configured as a multipolar, circular (e.g., pigtail) catheter.

FIG. 10 is an example of a temperature-sensing catheter configured with a paddle-shaped distal end region including the array of (multipolar) electrodes and temperature sensors.

### DETAILED DESCRIPTION

The methods, devices and systems described herein can be used to detect and monitor the temperature of portions of the heart in diagnosis and/or treatment of cardiac arrhythmia. Such conditions, may include any arrhythmia, such as supraventricular tachycardia (SVT), Wolff-Parkinson-White (WPW) syndrome, atrial fibrillation, atrial flutter, atrial tachycardia, and ventricular arrhythmias. One example of SVT is AV-nodal reentrant tachycardia (AVNRT), which is associated with a re-entrant circuit near the AV node, the His bundle and the coronary sinus.

In some implementation, the methods and devices are used during a catheter ablation procedure, which involves insertion of an ablation catheter to the patient's heart and to an ablation target associated with the arrhythmia. Typically, a mapping catheter is used to record the heart's electrical activity and to pinpoint the origin of the arrhythmia. The area of the heart muscle at the affected site is then destroyed/removed, or modified using the ablation catheter to cause localized heating (e.g., radiofrequency ablation) or freezing (e.g., cryoablation) of the target tissue. This creates scar tissue at the affected site, which prevents the aberrant electrical signals from affecting normal electrical conduction of the heart.

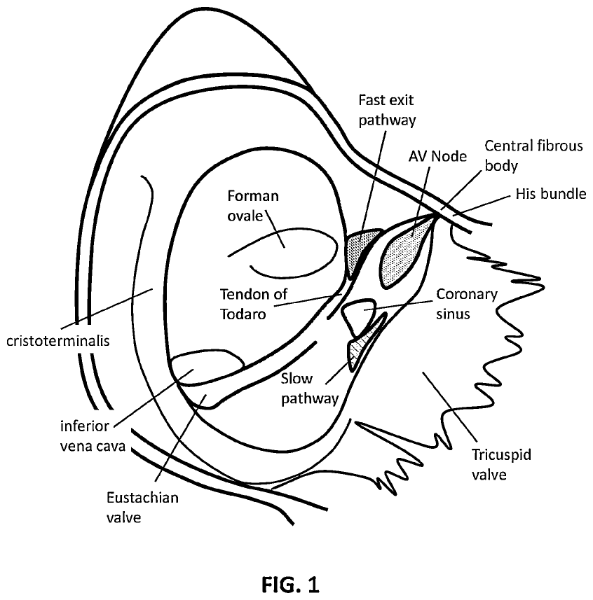

FIG. 1 shows a view of the Koch's triangle portion of a heart illustrating an example of an AVNRT re-entrant circuit near the AV node. In this example, the AVNRT is associated with a slow pathway (adjacent to the coronary sinus) and fast pathway exit near the AV node, and the His bundle. The electrical signals through the slow pathway and fast pathway exit can cause the heart to beat abnormally fast, resulting in heart palpitations. In typical AVNRT, the anterograde conduction is via the slow pathway and the retrograde conduction is via the fast pathway (“slow-fast” AVNRT). In atypical AVNRT, the anterograde conduction is via the fast pathway and the retrograde conduction is via the slow pathway (“fast-slow” AVNRT). Multiple slow pathways may exist so that both anterograde and retrograde conduction are over slow pathways (“slow-slow” AVNRT).

Treatment for AVNRT can include catheter ablation of a portion of the AVNRT circuit, typically at the slow pathway. Catheter ablation generally involves applying thermal treatment (e.g., via RF heat catheter or cryotherapy catheter) to the target area of the heart to form scar tissue at the ablation site that interrupts the AVNRT circuit. However, target ablation sites for treating the AVNRT circuit is typically very near the AV node. For example, the slow pathway region of the AVNRT may be a distance of about 15-20 millimeters (mm) from the AV node. Thus, when the ablation catheter applies thermal treatment to one or more areas of the AVNRT circuit, there is a risk that the heat or cold may radiate through the tissue and damage the nearby AV node and/or other nearby portions of the heart. Such damage may cause AV node block, which can disrupt the patient's heart rhythm and may require implantation of an artificial pacemaker.

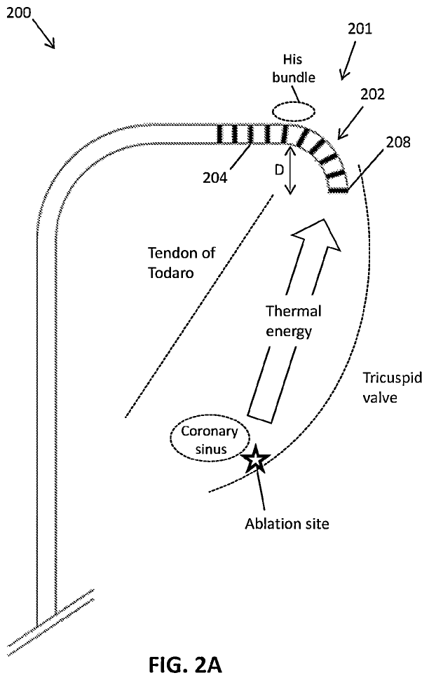

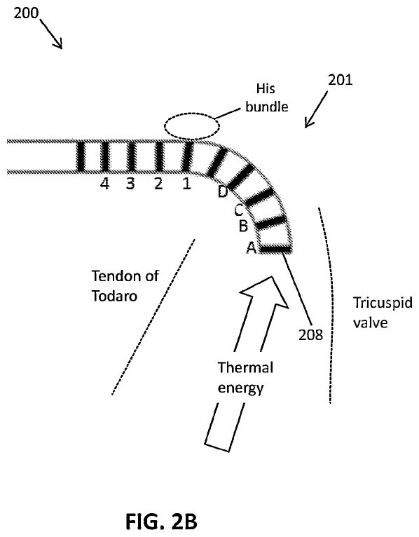

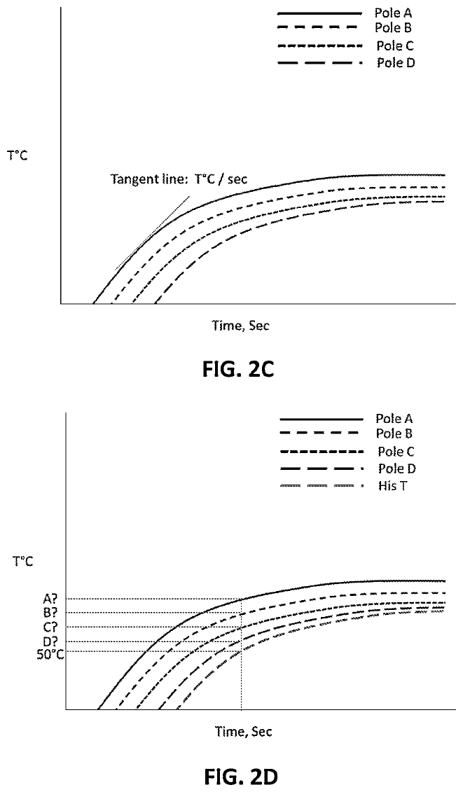

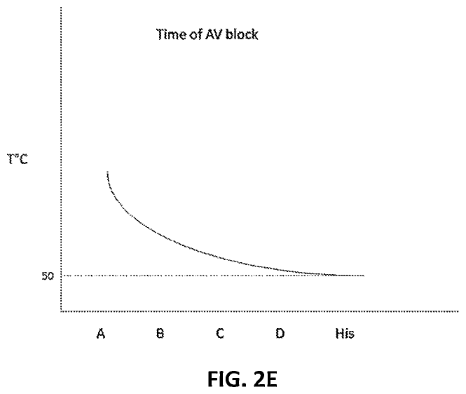

To address this and other issues related to catheter ablation, the methods described herein may be used to detect and monitor the temperature of heart tissue a distance from the ablation site during the ablation procedure. In the case of an AVNRT catheter ablation treatment procedure, the methods may involve monitoring regions of the heart at or near the AV node, the His bundle and/or the coronary sinus. In some cases, the methods are implemented using a temperature sensing catheter. FIG. 2A shows an example temperature sensing catheter 200, which includes a distal sensing portion 201 adapted to maneuver within passages of a subject's heart. The sensing portion 201 can include multiple EGM electrodes 204 adapted to detect electrical activity at an intra-cardiac surface. The EGM electrodes can be connected to wires within an inner lumen of the catheter body (shaft) and be coupled to a recording device and/or computer (e.g., controller) at a proximal end of the catheter. In some embodiments, one or more of the EGM electrodes is ring-shaped such that an external surface of at least one of the electrodes runs around the circumference of the catheter. The catheter can also include one or more temperature sensors (e.g., thermocouple, thermistor, infrared temperature sensor, fluoroptic temperature sensor and/or other temperature sensing devices), which are adapted to detect the temperature of at an intra-cardiac surface. In some embodiments, the one or more temperature sensors are situated along the catheter between or adjacent to the EGM electrodes. In some embodiments, the temperature sensors are configured to measure temperature radially around the circumference of the catheter for a 360 degree field of temperature sensing. In some cases, the temperature sensors are ring-shaped (e.g., ring sensors) in accordance with the shaft-shaped catheter. In some embodiments, the EGM electrodes themselves function as temperature sensors. For instance, voltage reading from the EGM electrodes can be used to calculate an impedance on the electrodes, which can then be used to determine the temperature on the electrodes based on the electrode material.

The temperature sensing catheter 200 can be adapted to measure the temperature and/or perform electrophysiological mapping of regions (e.g., perinodal locations) of the heart during an ablation procedure or diagnostically (e.g., not during an ablation procedure). In some implementations, the temperature sensing catheter 200 is used in conjunction with an ablation catheter for treating AVNRT, where the ablation catheter is used to ablate an ablation site near the coronary sinus (e.g., AVNRT slow pathway). Such ablation procedure can cause a transfer of thermal energy through other parts of the heart, including toward/away from the AV node. If the ablation is a heat-based ablation, the heat can dissipate toward the AV node, thereby risking damage the AV node by overheating. If the ablation is a cold-based ablation (cryoablation), the heat can dissipate away the AV node, thereby risking damage the AV node by overcooling. The AV node may be generally in a region near the His bundle. Thus, the temperature sensing catheter 200 can be position at or near the His bundle and toward the ablation site near the coronary sinus to detect the temperature at these regions during the catheter ablation procedure, thereby providing a way to determine whether the AV node becomes overheated or overcooled.

In practice, the temperature sensing catheter may be positioned within the heart using the EGM electrodes. The EGM electrodes may be moved along the conduction pathways and the inner walls of the heart, measuring electrical activity along the way to locate portions of the heart's electrical conduction system, such as the His bundle and/or AV node. Once properly positioned, the temperature sensors can be used to detect and monitor the temperatures before, during and/or after the ablation procedure. In some cases, multiple temperature sensing catheters can be used to detect and monitor the temperature of different regions of the heart. For example, a first temperature sensing catheter may be positioned at or near the His bundle and a second temperature sensing catheter may be positioned at or near the coronary sinus to monitor the temperatures in these regions during an AVNRT ablation procedure.

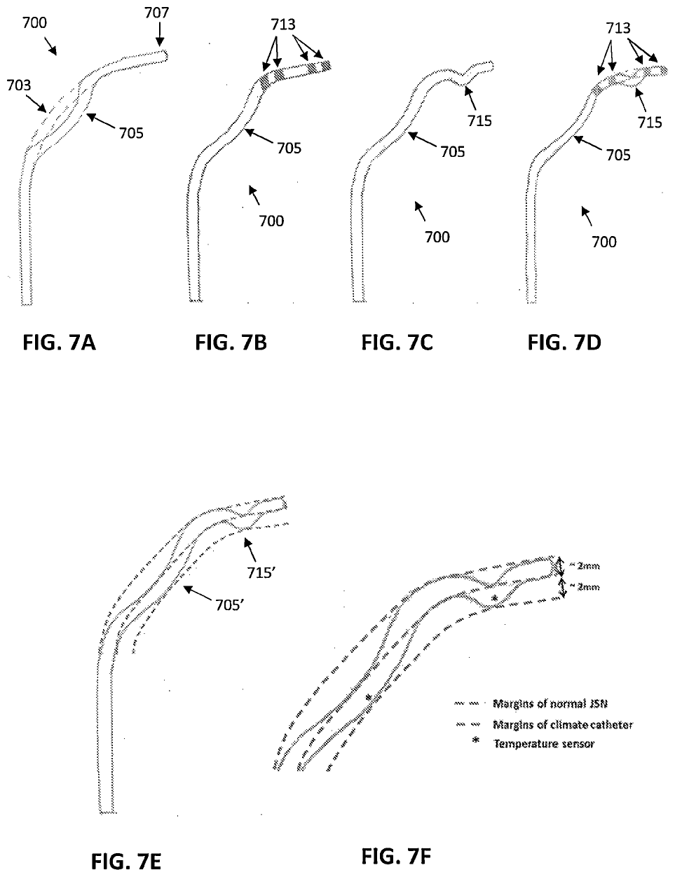

The distal sensing portion 201 of the catheter can include one or more curved portions 202, corresponding to a bend along a length of the catheter (note that FIGS. 7A-7F show additional examples of catheters, including temperature-sensing catheters, as described herein). The curved configuration can allow the EGM electrodes and/or temperature sensors of the distal sensing portion to come into contact with different regions of the heart. In the example shown in FIG. 2A, one or more of the temperature sensors of the curved portion 202 may be well positioned for measuring the temperatures at or near the His bundle, while one or more of the temperature sensors of the curved portion 202 (e.g., at the distal tip 208) may be well positioned for measuring the temperatures in regions of the heart closer to the ablation site. The curved portion may extend the distal tip 208 by a distance D in a direction toward the ablation site. The distance D may vary depending on a particular use case of the catheter diagnostics. In some embodiments, the distance D ranges from 5 millimeters (mm) to 15 mm (e.g., 5, 7.5, 8, 9.2, 9.55, 10, 12, 13.25 or 15 mm). The extent of curvature of the curved portion 202 may vary. In some embodiments, the curved portion 202 has a curvature ranging from 1 degree)(° and 270° (e.g., full circle) (e.g., 2, 5, 20, 45, 90, 180 or 270°). In some cases, the distal tip 208 is positioned a certain distance from the ablation site, which may vary based on the ablation procedure parameters and/or the particular anatomy of the subject. In some embodiments, the distance between the distal tip 208 and the ablation site ranges from about 1 centimeters (cm) to 2 cm.

The distal tip 208 may be deflectable or non-deflectable. In cases where the distal tip is deflectable, at least part of the distal portion 201 may be uni-directionally deflectable (e.g., in one direction in a plane), bi-directionally deflectable (e.g., in two directions in a plane), or multi-directionally deflectable (e.g., in three dimensions) to allow the user to steer the distal tip toward a particular location of heart, for example, toward the site of ablation. In some cases, the distal portion 201 can be deflectable from a straight configuration and bend by anywhere between 5 degrees)(° and 270° (e.g., full circle). The deflection may be controllable by the user at a proximal end of the catheter. In some cases, the distal portion 201 is fixed (not deflectable) and may include a fixed curve portion 202.

The distal sensing portion 201 can include any number of EGM electrodes and temperature sensors (e.g., 1, 2, 3, 4, 5, 6 etc.). In some cases, the quadrant diameter and radius of the catheter may vary based on the number of electrodes on the curved portion 202. Table 1 provides example quadrant diameter and radius for different number of EGM electrodes on a curved portion based on a 2 mm inter-electrode distance and 1 mm electrode width.

TABLE 1

| Electrodes in curve | Quadrant Diameter (mm) | Radius (mm) |
|---|---|---|
| 5 | 15 | 9.55 |
| 4 | 12 | 7.64 |
| 3 | 9 | 5.73 |
| 2 | 6 | 3.82 |

FIG. 2B shows a close-up view of the distal sensing portion 201. The distal sensing portion can include different combinations of EGM electrodes and/or temperature sensors 1, 2, 3, 4, A, B, C and D. For example, in some embodiments, 1, 2, 3 and 4 may correspond to EGM electrodes for detecting electrical activity of the heart while A, B, C and D may correspond to temperature sensors configured to detect temperature of the heart. In such a configuration, A, B, C and D that are distal to the His bundle may correspond to electrically insulated temperature sensing poles without an associated EGM electrode, thereby reducing a potential antenna effect on the EGM electrodes 1, 2, 3 and 4. In other embodiments, 1, 2, 3 and 4 may correspond to temperature sensors and A, B, C and D may correspond to EGM electrodes for detecting electrical activity. In other embodiments, 1, 2, 3, 4, A, B, C and D may each include a combination of temperature sensors and EGM electrodes. In some cases, a reference temperature sensor is located on a proximal site of the catheter. The spacing between the EGM electrodes may vary depending on signal accuracy requirements (e.g., for measuring a particular portion of the heart), with smaller spacings generally associated with higher signal accuracy. In some cases, the spacing between the EGM electrodes can range from 0.5 mm to 3 mm (e.g., 0.5, 1, 2, 3 mm) for a decapolar or octapolar catheter setup.

In some embodiments, readings from the multiple temperature sensors are used to create a gradient of temperatures related to the thermal activation during ablation at the distant site. For example, in embodiments where each of poles A, B, C and D of catheter 200 include temperatures sensors, the temperature readings from the poles A, B, C and D can detect varying temperatures depending on their proximity to the ablation site. FIG. 2C shows a graph indicating an example calculated rise in tissue temperature (T° C.) as a function of time for each of the poles A, B, C and D. As shown, the temperature reading at pole A at any particular time is greater than that pole B, which is greater than that pole C, which is greater than that pole D. This gradient information can be used to estimate the temperature of the His bundle and/or the AV node at various times during the ablation procedure. FIG. 2D is a graph showing the calculated rise in tissue temperature measured at each of the poles A, B, C and D compared to an estimated onset of injury to the AV node based on a temperature of the His bundle. The estimated onset of injury to the AV node may be a function of temperature at the AV node (e.g., 50° C.-56° C.), based on earlier studies. The calculated data indicates that the placement of poles A, B, C and D at different distances from the ablation site can be used to create a temperature gradient that can be used to calculate the rate and acceleration of temperature increases or decreases in the region near the AV node and/or His bundle. The temperature gradient can be compared to upper or lower threshold limits (e.g., at the His bundle) determined to create thermal injury to the AV node. Examples of temperature-sensing catheters that may be used to sense temperature are described below, including in FIGS. 7A-7F. In some examples more than one temperature-sensing catheter may be used.

In some embodiments, the catheter system can be configured to forewarn the user of the development of transient or permanent heart block (e.g., AV block) based on the readings from the temperature sensors. For example, the system may can be configured to determine at what pole and/or distance from the His bundle and/or AV node such temperature increases occur. In some cases, calculation are based on the measured reduction in unipolar signal on the poles distal to the His bundle or AV node signals that are positioned between the His/AV node and ablation site. In some cases, calculation are based on the measured reduction in electrode impedance on the poles distal to the His bundle or AV node signals that are aligned between the His/AV node and ablation site. The system may be configured to determine at which of the distal poles and at what temperature, temperature rate and/or temperature acceleration junctional speeding occurs during cardiac ablation. For example, junctional speeding refers to the desired outcome when doing AVNRT ablation; ideally the junctional speeding may be −500 ms. The rate of junctional speeding may be monitored and may be used as a potential indicator of a cardiac issue, such as AV block; in some examples junctional speeding following ablation of <400 ms rate of junctional rhythm may correlate with an impending AV block. In any of the methods and apparatuses described herein junctional speeding may be detected from an intracardiac electrogram (e.g., in real time). The apparatuses described herein may automatically or semi-automatically analyze the intracardiac electrogram (which may be collected from the one or more catheters described herein) and may indicate (e.g., by one or more displays, alarms, etc.) a rate (or acceleration of rate) of junctional speeding.

The apparatuses, e.g., systems, described herein may additionally or alternatively be configured to determine absolute temperature of each temperature sensor, identify the rate of thermal increase (or decrease) (T° C./sec) along the catheter, calculate an estimated time in which an AV block can occur (FIG. 2E) and/or identify the rate of thermal acceleration (or deceleration) (T° C./mm/sec) along the catheter. In cases where a second temperature sensing catheter is placed at a second location (e.g., at or near the coronary sinus), the temperature reading from the second catheter can be used to identify absolute, rate of increase and/or acceleration of temperature at the second location. The system may be additionally or alternatively be configured to calculate the acceleration of power at the temperature sensors, which can forewarn the development of transient or permanent AV block or other adverse event, and association with junctional speeding. Examples of adverse events may include damage to bundle branch, sinus node, etc. The system may be additionally or alternatively be configured to calculate the delivered energy at each temperature sensor, rate of energy increase (e.g., power) and acceleration for each temperature sensor, and/or absolute energy at each temperature sensor. Such calculations may be based on the Heat equation (Equation 1) and the heat transfer (Equation 2) below.

$$\frac{\partial u}{\partial t} = α ( \frac{{\partial }^{2} u}{\partial {x}^{2}} + \frac{{\partial }^{2} u}{\partial {y}^{2}} + \frac{{\partial }^{2} u}{\partial {z}^{2}} ) \qquad Equation 1$$

for a function μ(x,y,z,t) of three spatial variables (x,y,z) and the time variable t, where α is a real coefficient called the diffusivity of the medium.

$$\frac{Q}{t} = \frac{kA ( T 2 - T 1 )}{d} \qquad Equation 2$$

where k is the thermal conductivity of the material (e.g., k (heart muscle)=0.56), A is the surface area of objects in contact, T2−T1 is the different in temperature between two materials, and d is the distance between the materials.

Thus, the temperature gradient data can be used to calculate an estimated rate of increase or decrease in temperature, which in turn can be compared to threshold rates of increase or decrease to determine if and/or when temperatures at a region of interest may incur thermal injury. For example, a rate of temperature increase during a heat ablation can be compared to a threshold rate of temperature increase to determine if the rate of temperature increase is likely to cause thermal injury at a certain time. Likewise, a rate of temperature decrease during a cryothermal ablation can be compared to a threshold rate of temperature decrease to determine if the rate of temperature decrease is likely to cause thermal injury at a certain time. In some cases, the temperature gradient data can be used to calculate an estimated acceleration or deceleration in temperature, which in turn can be compared to a threshold acceleration or deceleration to determine if and/or when temperatures at a region of interest may incur thermal injury. In some cases, the temperature gradient data is compared to multiple thresholds corresponding to different injury severities. The temperature sensing catheter system may be configured to provide an alert (e.g., visible and/or audible alert) to the user to forewarn of the risk of injury. The system (e.g., automatically) or user (e.g., manually or semi-automatically) may then modify the ablation (e.g., stop or temporarily halt) or modify ablation parameters without stopping the ablation process, for example, by decreasing or increasing the intensity of the ablation, the ablation time and/or the area of ablation. In some cases, the user may control the temperature at the ablation site and/or at other, nearby sites, by delivering a fluid (e.g., water) to the heart or inserting a separate cooling or heating catheter near the region of interest.

For example, in some examples, the method and/or apparatus may deliver fluid and/or using a cooling or heating catheter to adjust the temperature of the ablation region and/or of the nearby tissue regions based on the temperature (e.g., based on the risk).

Any of the methods and apparatuses described herein may use one or more electroporation catheters. Electroporation catheters may be used for cardiac ablation (e.g., AF ablation, etc.). Electroporation catheters typically work by disrupting cell membranes (essentially via application of direct current) and may have a lower risk of causing collateral thermal injury to adjacent structures like oesophagus compared to other techniques. Thus, any of these method and apparatuses described herein, including temperature monitoring, may include or be used with an electroporation catheter for ablation.

In some cases, the temperature sensing catheter is configured to provide feedback data to a control system controlling the ablation process. For example, an ablation catheter system controlling the ablation process may be configured to receive feedback data from the temperature sensing catheter system and modify the ablation parameters (e.g., automatically) to prevent thermal injury to the region of interest. In some cases, the feedback data is used to stop (and/or pause) the ablation. In some cases, this involves causing the heating or cooling mechanism to turn off. In some cases, the feedback data is used to modify ablation parameters without stopping the ablation process, for example, by decreasing or increasing the intensity of the ablation, the ablation time and/or the area of ablation. In some embodiments, feedback from the temperature sensing catheter is used to automatically control a secondary device such a separate cooling or heating catheter.

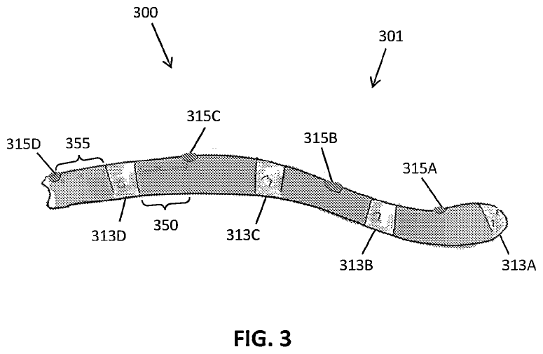

FIG. 3 shows a distal sensing portion 301 of another temperature sensing catheter 300. In this example, the distal sensing portion includes temperature sensors 315A, 315B, 315C and 315D (e.g., thermocouples, thermistors, infrared temperature sensors, fluoro-optic monitors and/or other temperature sensing devices) situated between ring-shaped EGM electrodes 313A, 313B, 313C and 313D. The catheter may be positioned within the heart such that the temperature sensors are oriented toward the cardiac tissue (e.g., in contact the cardiac tissue). In some embodiments, the temperature sensors protrude from the surface of the catheter body. In one example, the temperature sensors protrude from the surface of the catheter body by 0.1 mm to 1 mm (e.g., 0.1, 0.5, 0.75, 0.1 mm) from the catheter body. In some embodiments, the catheter has a curved distal sensing portion that may be deflectable or non-deflectable. In some cases, the catheter is configured for placement within and pacing of the right ventricle. The temperature sensors and EGM electrodes can be connected to wires within a lumen of the catheter body and terminate proximally at one or more pins and/or connectors (e.g., push connector) to a cable (e.g., decapolar cable). The temperatures sensors may be positioned uniformly between adjacent EGM electrodes (e.g., distance 350 is equal to distance 355) or non-uniformly between adjacent EGM electrodes (e.g., distance 350 is not equal to distance 355).

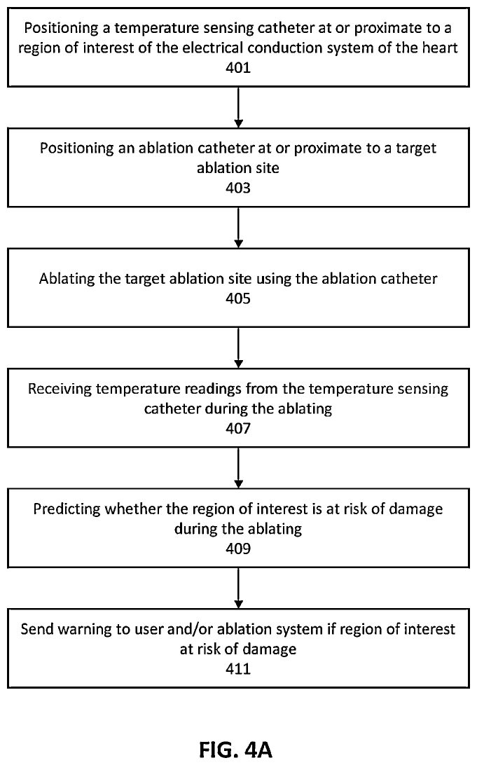

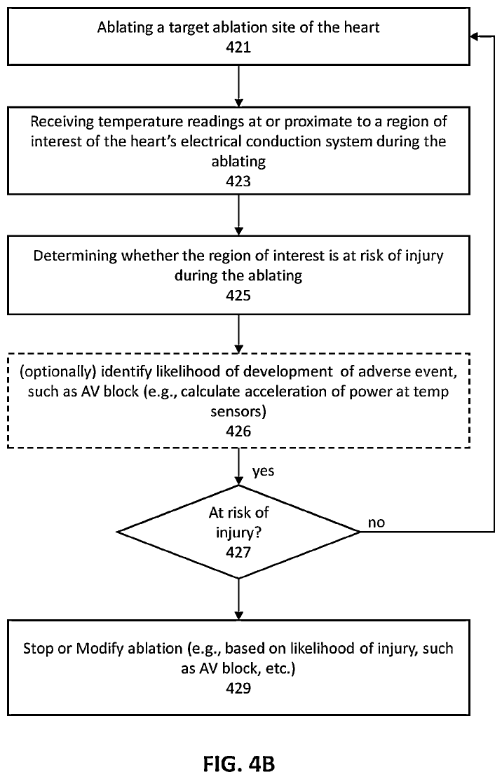

FIG. 4A shows a flowchart illustrating an example process for using a temperature sensing catheter to forewarn damage to a portion of the heart during an ablation procedure. The process includes positioning one or more temperature sensing catheters at or near one or more regions of interest of the electrical conduction system in the subject's heart 401. In some embodiments, such as for an AVNRT ablation procedure, a region of interest can include the AV node. In some embodiments, the positioning involves using EGM electrodes of the temperature sensing catheter to determine electrical activity (e.g., record one or more electrograms) along the endocardial surface and positioning a distal sensing portion of the catheter accordingly. For example, in the case of an AVNRT ablation procedure, the EGM electrodes may be used to guide the distal sensing portion of the catheter at or near the His bundle or the coronary sinus. In some cases, the positioning includes directing a distal tip of the temperature sensing catheter toward a target ablation site for the ablation procedure. For example, a curved distal sensing portion of the temperature sensing catheter may be positioned (e.g., deflected) such that the distal tip is generally pointed toward and is the closest portion of the temperature sensing catheter to the target ablation site. Temperature readings from temperature sensors along the distal sensing portion can be used to create a temperature gradient along a path from a location of temperature sensing catheter toward the target ablation site, where the temperature sensors closer to the target ablation site have higher temperature readings than temperature sensors farther from the target ablation site.

At 403, an ablation catheter is positioned at or proximate to the target ablation site. In the case of an AVNRT ablation procedure, the target ablation site may include a slow pathway and/or a fast pathway exit of the AVNRT. Typically, a mapping catheter is used to guide and position the ablation catheter to the target ablation site. The ablation catheter can then be used to ablate the target ablation site 405. The ablation may include heating the target ablation site or cooling the target ablation site (cryotherapy). Temperature readings can be received during the ablation from the temperature sensing catheter 407. The temperature readings can be used to calculate a number of factors related to the ablation procedure, including one or more of: the absolute temperature at each of the temperature sensors; the distance of each of the temperature sensors from the region of interest (e.g., AV node), the ablation site and/or other portions of the heart (e.g., His bundle or coronary sinus); the unipolar signal activation attenuation rate and/or impedance (+/−) at each of the temperature sensors; the rate of thermal increase/decrease and/or thermal acceleration/deceleration along the catheter; the energy and/or power at each of the temperatures sensors; the acceleration of power at each of the temperatures sensors; and an estimated time in which the damage will occur to the region of interest (e.g., AV block). In some cases, this information is displayed to the user, for example at a user interface (e.g., display screen). In some instances, the highest recorded temperature (and/or the calculated energy and power) of the multiple temperature sensors per EGM electrode is displayed. In some implementation, certain parameters, such as spread and/or acceleration of temperature or energy/power is displayed on a 3D geometric surface model.

The calculated information can be compared to threshold values (e.g., upper/lower threshold temperatures or rate of temperature increase/decrease) to predict if and when the region of interest is at risk of damage during the ablation procedure 409. If multiple temperature sensing catheters are used, data from the multiple temperature sensing catheters can be used to predict if and when the region of interest is at risk. If it is determined that the region of interest is at risk at any time during the ablation procedure, the system can send a warning (e.g., visual and/or audible alarm) to the user and/or to the ablation catheter system 411.

FIG. 4B shows a flowchart illustrating an example process for using a temperature sensing catheter with feedback control. At 421, a target ablation site is ablated using, for example, an ablation catheter. In the case of an AVNRT ablation procedure, the target ablation site may include a slow pathway and/or a fast pathway exit of the AVNRT. At 423, temperature readings at or proximate to a region of interest of the heart's electrical conduction system during the ablation procedure. In the case of an AVNRT ablation procedure, the region of interest may include the AV node. The temperature readings may be detected by a temperature sensing catheter that includes multiple temperature sensors along the length of the catheter body. The temperature readings may include temperature gradient data based on temperature differences among the different temperature sensors and their relative distances from the ablation site. The temperature gradient data can be used to calculate increases and decreases in temperature, energy, and/or power at the region of interest, and acceleration and deceleration of in temperature, energy, and/or power at the region of interest. This data can be compared to thresholds (e.g., upper and lower temperature, upper and lower rate of increase/decrease in temperature, and/or upper and lower temperature acceleration/deceleration). In some cases, the threshold values may be predetermined based on, for example, experimental and/or calculated data associated with thermal damage to the region of interest. At 425, the information can be used to determine whether and/or when the region of interest is at risk of thermal damage during the ablation procedure. These methods and and/or apparatuses for performing them may also or alternatively identify the likelihood of adverse events due to the procedure, such as but not limited to AV block, damage to bundle branch, sinus node, etc. 426. In some examples this may include calculating a likelihood (e.g., percent, index, etc.) that may be compared to a range or threshold and used to determine the risk of injury and/or the procedural effect (e.g., stopping or modifying the applied energy).

If is it determined that the risk of damage is within an acceptable range (e.g., below a threshold, below an upper threshold and/or above a lower threshold), the ablation process may be allowed to proceed 421. If it is determined that the risk of damage is outside of an acceptable range (e.g., at or above an upper threshold, or at or below the lower threshold), this information can be used as feedback to modify the ablation process 429. The feedback may include stopping the ablation (e.g., if the risk is above a stopping threshold; the feedback may include modifying the ablation, e.g., if the risk is below the stopping threshold, but above the risk threshold. For example, if the rate of increase of the energy is low, the energy applied may be adjusted to reduce the rate of increase and/or maintain or reduce the temperature at some or all regions, thereby lowering the risk of adverse events.

In some cases, the feedback control is automatic. In some embodiments, the feedback may be to distal connector and sensing system. In some embodiments, the feedback may be integrated with an ablation system and/or mapping system associated with the ablation catheter. In some implementations, the feedback is used to stop the ablation process, e.g., by turning off the heating or cryothermal energy. This may be used if it is determined that critical limits (e.g., maximum temperature) have been reached or are predicted to be reached within a period of time. In some implementations, the feedback is used to modify ablation parameters without stopping the ablation process. For instance, the feedback data may be used to titrate energy delivery from the ablation catheter for a typical response required, as in the case of junctional acceleration during ablation of AVNRT.

In some embodiments, the system is configured to predict whether and/or when a region of interest is at risk of damage before the ablation procedures is performed. For example, ablation parameters (e.g., power, distance from the target ablation site) of the ablation catheter may be used to determine whether and when such ablation parameters may put the region of interest at risk of injury. The ablation parameters may then be adjusted based on this determination.

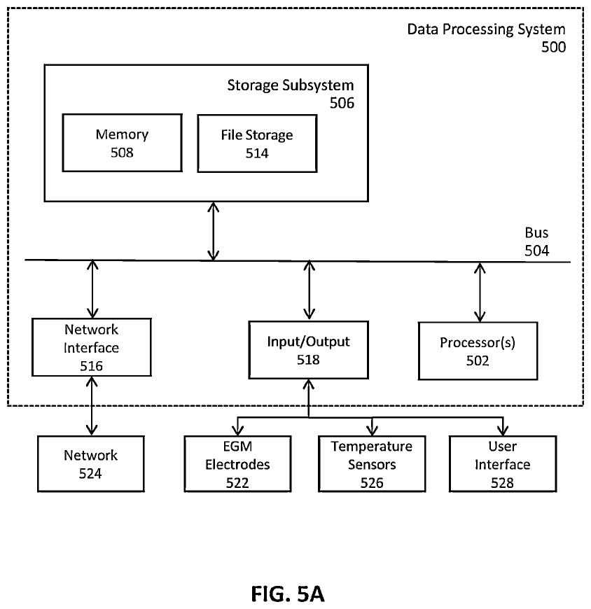

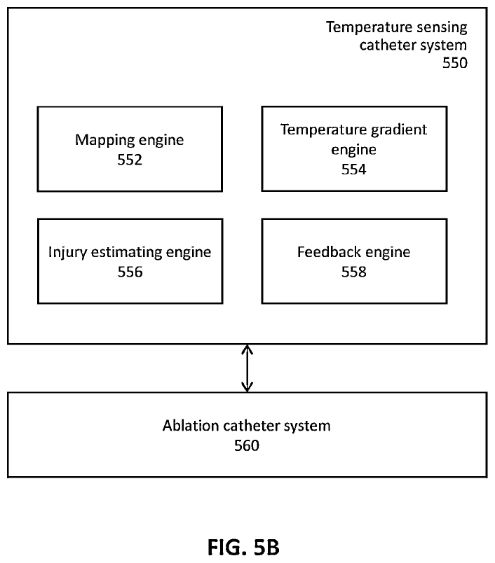

The methods described herein may be performed by a data processing system, which may include hardware, software, and/or firmware for performing one or more of the methods described herein. FIG. 5B shows a simplified block diagram of an example data processing system 500 for a temperature sensing catheter. Data processing system 500 typically includes one or more processors 502, which communicates with a number of peripheral devices over a bus 504. These peripheral devices typically include a storage subsystem 506, one or more input and/or output interfaces/devices 518, and a network interface 516. System 500 may include a controller for a diagnostic catheter computing device, a terminal or personal computer, a workstation, a mainframe and/or may be integrated with an ablation catheter system.

The processor(s) 502 can be configured to implement one or more of the methods described herein. In some embodiments, the processor(s) can be configured to map the location of the sensing portion of the catheter based on input from the EGM electrodes. In some embodiments, the mapping is in three-dimensions (3D). The user interface can be configured to render (e.g., via a display) the mapping (e.g., in 3D). In some embodiments, the processor(s) can be configured to calculate a gradient of temperatures based on input from multiple temperature sensors of the catheter. The temperature gradient can be correlated spatially with the mapping information to estimate a directionality of temperature conduction.

The storage subsystem 506 can be configured to maintain basic programming and data constructs that provide the functionality of the devices and methods described herein. The storage subsystem may store software modules for implementing one or more of the methods described herein. In some embodiments, the storage subsystem 506 includes memory 508 and file storage 514. Memory 508 may include a number of memories including a main random access memory (RAM) for storage of instructions and data during program execution and a read only memory (ROM) in which fixed instructions are stored.

The input/output interface 518 can may include one or more user interface input devices and/or one or more user interface output devices. The user interface input device(s) may include a touch pad, keyboard, pointing device and/or other input device. The pointing device may be an indirect pointing device such as a mouse, trackball, touchpad, or graphics tablet, or a direct pointing device such as a touchscreen incorporated into the display. Other types of user interface input devices, such as voice recognition systems, may be used. The user interface output device(s) may include a display device, printer and/or other output device. The display device may be a flat-panel device such as a liquid crystal display (LCD), a cathode ray tube (CRT) or a projection device. The display device may provide nonvisual display such as audio output. In some cases, the display includes a display subsystem, which may include a display controller and a display device coupled to the display controller.

The network interface 516 can be configured to couple with corresponding interface devices for one or more networks 524 (e.g., local network, wide area network, cloud and/or the Internet). The file storage 514 can be configured to provide persistent (nonvolatile) storage for program and data files, and may typically include at least one hard disk drive and at least one removable media. There may also be other devices such as a CD-ROM drive and optical drives (all with their associated removable media). One or more of the drives may be located at a remote location, such as in a server on a local area network or at a site on the Internet.

In this context, the term “bus” is used generically so as to include any mechanism for letting the various components and subsystems communicate with each other as intended. The components coupled to the bus need not be at the same physical location. Thus, for example, portions of the file storage system could be connected over various local area or wide area network media, including telephone lines. Similarly, the input devices and display need not be at the same location as the processor(s). Bus 504 is shown schematically as a single bus, but a typical system may have a number of buses such as a local bus and one or more expansion buses (e.g., ADB, SCSI, ISA, EISA, MCA, NuBus, or PCI), as well as serial and parallel ports. Network connections may be established through a device such as a network adapter on one of these expansion buses or a modem on a serial port. The client computer may be a desktop system or a portable system.

In another embodiment, the system 500 of FIG. 5A can include a non-transitory computing device readable medium having instructions stored thereon that are executable by a processor to cause a computing device to receive data from a temperature sensing catheter during diagnosis of a subject's heart, which can be during an ablation procedure or separate from an ablation procedures (e.g., not during an ablation procedure).

FIG. 5B shows a simplified block diagram of a computing environment for a temperature sensing catheter system 550. The temperature sensing catheter system 550 can include one or more engines for performing one or more processes or methods described herein. As used herein, any “engine” may include one or more processors or a portion thereof. The system may include a mapping engine 552 for determining a location of a temperature sensing catheter based on received output from one or more EGM electrodes of the catheter. The system may include a temperature gradient engine 554 for calculating a temperature gradient based on received output from one or more temperature sensors of the temperature sensing catheter. The system may include an injuring estimating engine 556 for determining if and when injury to a region of interest of the heart may occur based, for example, on calculated data from the temperature gradient engine, the mapping engine and/or other data collected or calculated by the system. The system may include a feedback engine for providing feedback to the temperature catheter system 500 or another system, such as an ablation catheter system 560. In some cases, one or more aspects of the ablation catheter system can be controlled based on output from the feedback engine, such as stopping, pausing or otherwise modifying ablation parameters of an ablation catheter. In some embodiments, the ablation catheter system is configured to provide input to the temperature sensing catheter system.

### Example 1: Testing of Temperature Sensing Catheter

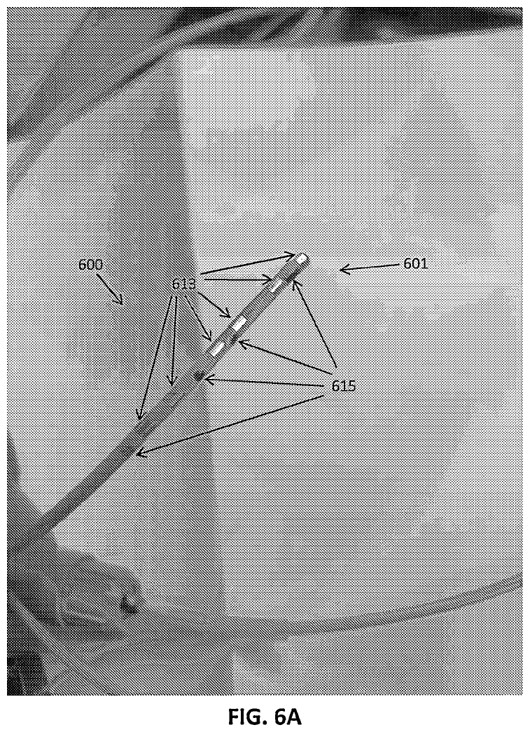

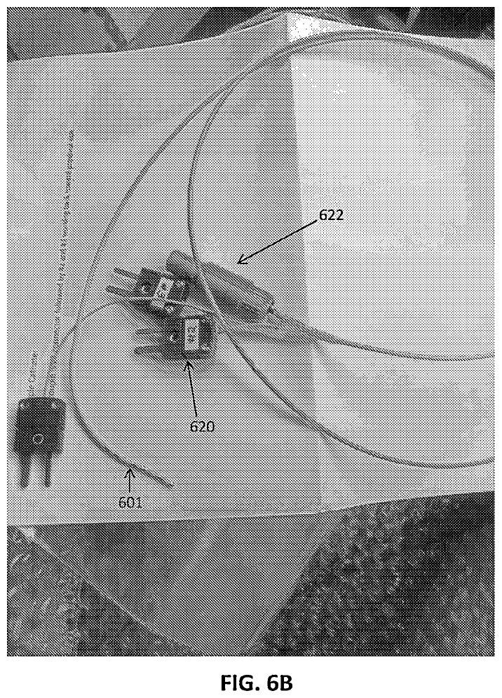

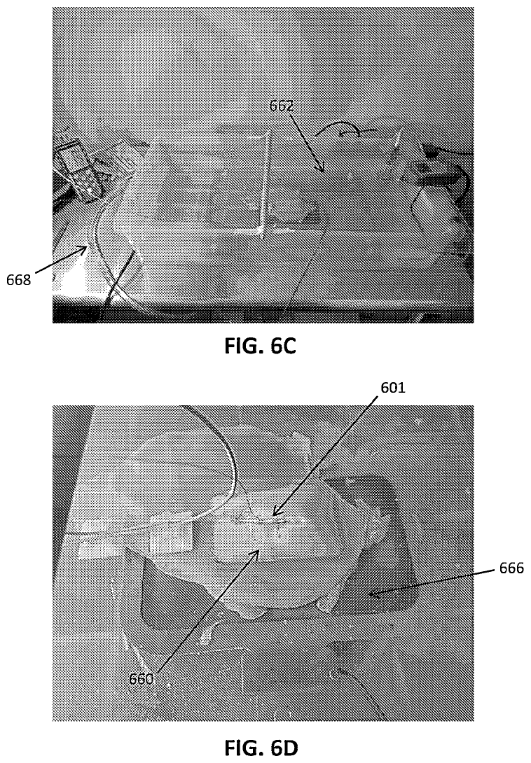

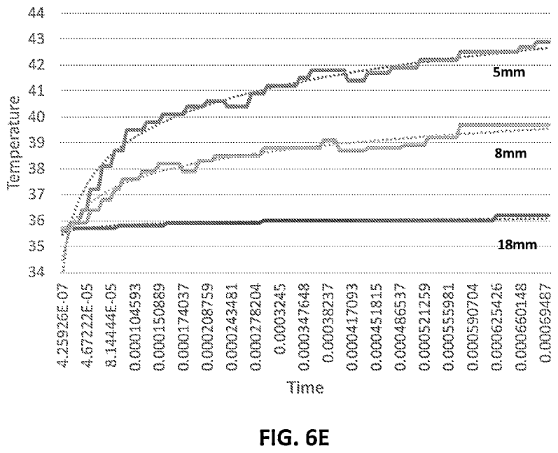

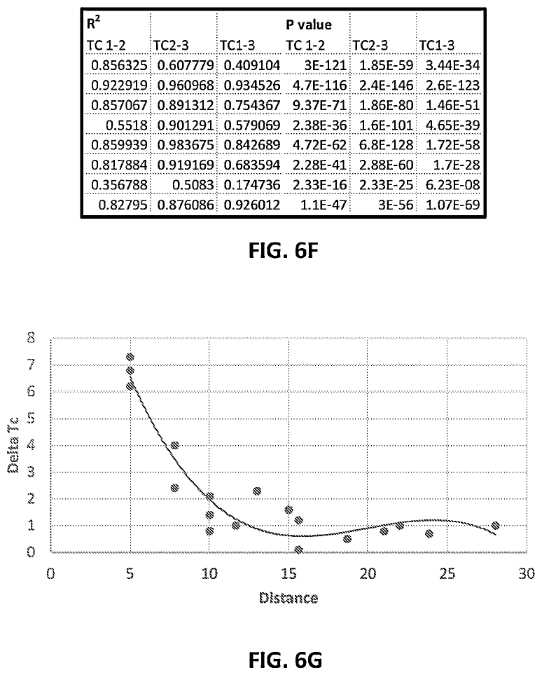

FIGS. 6A-6D show an example test setup for a temperature sensing catheter according to some embodiments. In this example, the distal sensing portion 601 of the temperature sensing catheter 600 includes six ring-shaped EGM electrodes 613 and four insulated thermocouples 615. The EGM electrodes are connected to wires within the catheter that lead to a first connector 622 at the proximal end of the catheter. The thermocouples are connected to wires within the catheter that lead to a number of second connectors 620 at the proximal end of the catheter. The catheter is fixed to a muscle tissue sample 660 such that the EGM electrodes and thermocouples are in contact with the muscle tissue sample. The assembly is placed in a temperature controlled water bath 662 with the muscle tissue sample placed on a return electrode 666 with ablation catheter applied to the opposing surface of the muscle to simulate a heating ablation procedure at a distal location. The option of fluid irrigation is provided via tubing 668 connected to the bath. The testing includes measuring temperature readings from the thermocouples while the bath is at different temperatures with and without irrigation.

Gradient in temperature appears to decrease as a polynomial function (probably partial differential equation) of distance from site of ablation. Regression analysis reveals a strong correlation of change in temperature over time between all thermocouples, with R2 values generally greater than 0.9 and all p values approaching zero.

FIG. 6E shows a graph indicating typically heating for a non-irrigated test during period of maximum temperature increase at different distances. FIG. 6F shows a table indicating correlation between different thermocouples during heating for non-irrigated ablation. FIG. 6G shows a graph indicating an example of change in temperature (maximum-minimum) versus distance from ablation site for a non-irrigated test.

As mentioned above, any of the method and apparatuses described herein may include one or more temperature-sensing catheters that include a plurality of temperature sensors and may be further configured to assume one or more shapes. For example, a temperature-sensing catheter may have a distal sensing portion with a distal tip. The distal sensing portion may include a plurality of temperature sensors (e.g., two or more, three or more, four or more, five or more, six or more, etc.) along a length of the distal sensing portion. These temperature sensors may be oriented to measure temperatures of an intra-cardiac surface. In some examples the sensors may extend circumferentially around or partially around (e.g., 25% or more of the way around, 30% or more of the way around, 40% or more of the way around, 50% or more of the way around, 60% or more of the way around, 75%, etc.) the circumference of the catheter each.

Any of the temperature-sensing catheters may be configured to assume a shape, e.g., a shape having one or more curves (“a curved shape”). For example, in some examples the temperature-sensing catheter may be biased (e.g., shape set) to assume one or more curves, e.g., at the distal end (the distal sensing portion, optionally including the distal tip region). Alternatively or additionally, the temperature-sensing catheter may be configured to include one or more members adapted to transition from a first shape to a second (e.g., curved, bent, etc.) shape. For example, the apparatus may be configured to include one or more tendons, wires, cables, etc. that may apply force on all or a portion of the length of the temperature-sensing catheter to bend the catheter. Thus any of these temperature-sensing catheter may be configured to have a curved shape such that the distal sensing portion can be oriented toward an ablation site within the heart during a cardiac ablation.

For example, these catheters may include a distal curved region (with or without active deflection). In some examples the catheters may include a modified inner bend region, e.g., including examples having one or more additional bends in combination with a curved distal end region (e.g., a modified inner bend region).

FIGS. 7A-7F illustrate examples of temperature-sensing catheters that are configured to assume a curved shape. FIG. 7A shows a first example of a temperature-sensing catheter 700 that is curved to the right along a first curve 703 (shown by the dashed lines), but may be further or alternatively deflected into a second curve 705 (shown by the solid lines). The first and/or second curve may be preset into the distal end region of the temperature-sensing catheter, or the first and/or second curves may be controllably formed, e.g., by actuating a tendon (e.g., pull-wire, cord, etc.) or other actuator (e.g., hydraulic, magnetic, piezoelectric, etc.) to deflect the elongate length of this region of the temperature-sensing catheter into the first and/or second curve 705. FIG. 7B shows the temperature-sensing catheter of FIG. 7A with the temperature sensors 713 included. In this example, the temperature sensors each extend circumferentially (shown as completely circumferentially, but all or some of these temperature sensors may be partially circumferentially around the temperature-sensing catheter, e.g., 25% or more, etc.).

Any of these temperature-sensing catheter may include additional regions of curvature. For example, FIGS. 7C and 7D show examples in which the temperature-sensing catheter incudes a third curved region 715 that may be preset or actuated, in addition to those shown in FIGS. 7A-7B. When all or some of these curves are controllable formed, these controllably curved regions may be separately or collectively (in any sub-combination) actuated. For example, different controls (e.g., tendons, wires, etc.) may be used to actuate individual or groups of curves.

FIGS. 7E and 7F illustrate another example of a temperature-sensing catheter similar to that shown in FIGS. 7C-7D, showing the temperature-sensing catheter in which the distal end region includes two curved portions 705′, 715′ in which the temperature-sensing catheter (also referred to herein as a climate catheter) temperature-sensing catheter transitions from a single arcing curve into a compound curve shape. This transition extends the margin of the temperature-sensing catheter to the right in this example, which, in use in a heart, may help drive the temperature-sensing catheter in proximity to the tissue for which the temperature will be sensed (e.g., offset from the ablation target tissue being treated).

The methods and apparatuses for cardiac ablation and/or monitoring described herein may include one or more temperature-sensing catheters in which the catheter comprises a single shaft. The catheter may be configured for multipolar mapping (e.g., may be configured as a multipolar mapping catheter). These catheters may be configured in a circular, paddle or lattice configuration, or some example of these configurations. In some examples the catheter comprises multiple prongs.

In general, these multipolar temperature-sensing catheters may be configured to maximize the surface area of tissue contact for temperature sensing in a region of interest, and may therefore assess a thermal gradient over this region.

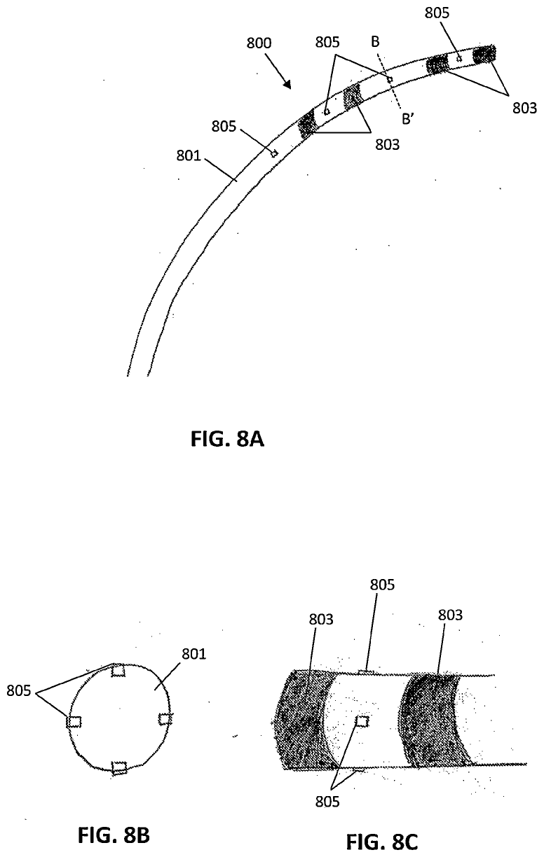

For example, FIG. 8A shows a first example of a distal end region of a temperature-sensing catheter having a linear, straight configuration. In this example, the catheter 800 includes a plurality of electrodes 803 spaced along the longitudinal length of the body 801 of the catheter, with one or more (e.g., a plurality of radially-arranged) temperature sensors 805 between the electrodes, as shown. FIG. 8B shows a cross-section through the catheter distal end region of FIG. 8A (through line B-B′ at the level of the temperature sensor(s)), and FIG. 8C shows an enlarged view of the distal end region of the catheter. In this example, the temperature sensors are arranged to stand proud of the outer surface of the catheter. In some examples the temperature sensors are flush (flat) with the outer surface of the catheter. In some examples the temperature sensors may be recessed relative to the outer surface of the catheter.

In some examples, having the temperature sensor (e.g., thermocouple, etc.) extend proud of the catheter body may be beneficial, e.g., to improve sensor-tissue contact for temperature sensing. Alternatively in some examples, having the sensor flush with the outer surface of the catheter may enhance smoothness and delivery of the catheter.

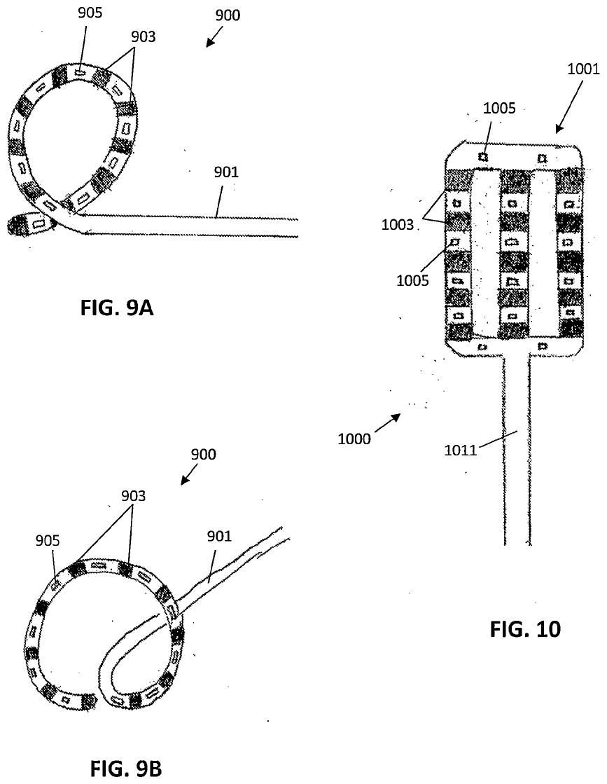

FIGS. 9A-9B illustrate examples of multipolar, circular catheters 900, which may also include electrodes 903 (arranged radially around the catheter body 901) and alternating with one or more temperature sensors 905. In some example, the catheter may be configured to switch between a straight configuration and a circular or curved configuration, for example, by longitudinally moving a stiffening member (e.g., central rod, wire, etc.) in/our of a lumen in the catheter and/or by pulling or actuating a pullwire or pullwires (not shown).

Although in FIGS. 8A-8C and 9A-9B the catheter includes multiple discrete temperature sensors at each level (longitudinal length) of the catheter, in some examples a single band or ring (e.g., ring-shaped) temperature sensor may be used or fewer or more than four temperature sensors may be used. For example, in some examples each of the two or more temperature sensors may be separately addressable/readable and may provide an oriented direction of thermal gradient; alternatively or additionally, in some examples groups of two or more temperature sensors may be combined (e.g., averaged, etc.) to provide a more robust temperature measure.

FIG. 10 illustrates another example of an apparatus 1000 for cardiac ablation and/or monitoring as described herein, configured to have a paddle shaped body 1001. In FIG. 10 a plurality of electrodes 1003 are arranged in an array over the paddle-shaped body, which extends from an elongate member 1011. A plurality of temperature sensors 1005 are arranged over the paddle-shaped body.

In any of the apparatuses described herein the temperature sensors (e.g., thermocouples) may be insulated to avoid electrical interference, whilst allowing heat transfer and temperature sensing. For example, electrically insulating material may be placed around and/or over the thermal sensors. The electrically insulating material may be thermally insulating and/or may be thermally conducting and electrically insulating (such as boron nitride nanosheets materials). In some examples an electrical insulation over the thermal sensor may reduce or avoid electrical noise during ablation affecting the temperature sensor.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of performing a cardiac ablation, the method comprising:
ablating a region of the heart to remove or modify cardiac tissue contributing to an arrhythmia;
measuring temperature at one or more locations in the heart that are at a distance from the ablation site during the ablating; and
predicting a risk that the heart will develop a transient or permanent atrioventricular (AV) block based on the measured temperature.

2. The method of claim 1, wherein ablating the region of the heart comprises ablating at least portion of the AV-nodal reentrant tachycardia (AVNRT) pathway, an AV re-entrant tachycardia pathway, an atrial tachycardia, and/or an atrial fibrillation or ventricular arrhythmia.

3. The method of claim 1, wherein the one or more locations in the heart includes at or near the His bundle, specialized conduction tissue or the coronary sinus.

4. The method of claim 1, further comprising measuring multiple temperatures along a path from the one or more locations toward the ablated region of the heart, and determining a temperature gradient along the path based on the multiple temperatures.

5. The method of claim 4, further comprising calculating increases or decreases of temperature, energy or power over time based on the temperature gradient.

6. The method of claim 5, wherein predicting the risk that the heart will develop a transient or permanent atrioventricular (AV) block includes determining whether one or more of the increases or decreases of temperature, energy or power reach or surpass an upper or lower threshold rate.

7. The method of claim 4, further comprising calculating accelerations or decelerations of temperature, energy or power based on the temperature gradient.

8. The method of claim 7, wherein predicting whether the AV node is at risk of developing a transient or permanent AV block includes determining whether one or more of the accelerations or decelerations of temperature, energy or power reach or surpass an upper or lower threshold acceleration or deceleration.

9. The method of claim 4, further comprising displaying the temperature gradient.

10. A method of performing a cardiac ablation, the method comprising:
positioning an ablation catheter to ablate a target ablation site in a subject's heart;
positioning a temperature sensing catheter to sense temperature at a region of interest separate from the target ablation site in the subject's heart;
ablating the target ablation site using the ablation catheter;
receiving temperature readings from the temperature sensing catheter during the ablating; and
predicting a risk of an adverse event based on the temperature readings; and
displaying the predicted risk and/or adjusting the ablation of the target ablation site based on the predicted risk.

11. The method of claim 10, wherein positioning the temperature sensing catheter includes directing a sensor region of the temperature sensing catheter toward the target ablation site such that the temperature sensing region is closest to the target ablation site.

12. The method of claim 10, wherein the risk of the adverse event comprises a transient or permanent heart block, or damage to a heart structure separate from the target ablation site.

13. The method of claim 10, wherein positioning the temperature sensing catheter includes deflecting a distal tip of the temperature sensing catheter toward the target ablation site.

14. The method of claim 10, wherein predicting the risk of includes determining whether one or more of the temperature readings reach or surpass an upper or lower threshold temperature.

15. The method of claim 10, wherein receiving temperature readings includes receiving the temperature readings from corresponding temperature sensors along a length of the temperature sensing catheter.

16. The method of claim 15, further comprising determining a temperature gradient along a path from the region of interest toward the target ablation site.

17. The method of claim 15, further comprising calculating increases or decreases of temperature, energy or power at or proximate to the region of interest over time.

18. The method of claim 17, wherein predicting the risk includes determining whether one or more of the increases or decreases of temperature, energy or power reach or surpass an upper or lower threshold rate.

19. The method of claim 15, further comprising calculating accelerations or decelerations of temperature, energy or power at or proximate to the region of interest.

20. The method of claim 19, wherein predicting the risk includes determining whether one or more of the accelerations or decelerations of temperature, energy or power reach or surpass an upper or lower threshold acceleration or deceleration.

21. The method of claim 10, further comprising determining that the region of interest is at risk of developing the transient or permanent heart block.

22. The method of claim 21, further comprising modifying the temperature at the region of interest based on the determining.

23. The method of claim 10, wherein receiving the temperature readings comprises receiving voltage readings from electrogram mapping (EGM) electrodes of the temperature sensing catheter, calculating impedance on the EGM electrodes based on the voltage readings, and calculating temperature on the EGM electrodes based on the calculated impedance and a material of the EGM electrodes.

24. A temperature sensing cardiac catheter system, comprising:
a catheter having a distal sensing portion with a distal tip, the distal sensing portion including a plurality of temperature sensors along a length of the distal sensing portion and oriented to measure temperatures of an intra-cardiac surface, wherein at least a portion of the catheter has a curved shape such that the distal sensing portion can be oriented toward an ablation site within the heart during a cardiac ablation; and
a non-transitory computer-readable medium including contents that are configured to cause one or more processors to perform a method comprising:
receiving temperature readings from the temperatures sensors; and
calculating a temperature gradient based on different temperature readings from the temperature sensors along the distal sensing portion.

25. The system of claim 24, wherein the plurality of temperature sensors include one or more of: a thermocouple, a thermistor, an infrared temperature sensor, and a fluoroptic temperature sensor.

26. The system of claim 24, wherein the temperature sensors are configured to measure temperature radially around the circumference of the catheter.

27. The system of claim 24, wherein the distal sensing portion is deflectable to assume a shape.

28. The system of claim 24, wherein the processor is configured to display the temperature gradient on a computer screen to a user.

29. The system of claim 28, wherein the non-transitory computer-readable medium is further configured to control the activity of an ablation catheter based on the temperature gradient.

30. The system of claim 28, wherein the non-transitory computer-readable medium is further configured to cause the processor to display the temperature gradient on a three-dimensional model including electro-anatomic mapping system and fluoroscopy system.

31. The system of claim 24, wherein the distal sensing portion further includes electrogram mapping (EGM) electrodes along the length of the distal sensing portion, the EGM electrodes configured to facilitate positioning of the distal sensing portion within the heart based on electrical signals of the heart.

32. The system of claim 31, wherein the EGM electrodes are separate from the temperature sensors.

33. The system of claim 31, wherein the EGM electrodes are the same as the temperature sensors.

34. The system of claim 24, wherein the catheter is not an ablation catheter.

35. The system of claim 24, wherein the catheter comprises a multipolar catheter.

36. The system of claim 24, wherein the catheter comprises a circular orientation.
